(19) United States
(12) Patent Application Publication
(10) Pub. No.: US 2022/0233549 A1
(43) Pub. Date: Jul. 28, 2022

(54) METHOD FOR ALPHAVIRUS INHIBITION

(71) Applicant: Venatorx Pharmaceuticals, Inc, Malvern, PA (US)

(72) Inventors: Glen A. COBURN, Bethel, CT (US); Guo-Hua CHU, Exton, PA (US); Jodie C. HAMRICK, New Holland, PA (US); Charlotte L. BOWSHER, Phoenixville, PA (US); Daniel C. PEVEAR, Downingtown, PA (US)

(21) Appl. No.: 17/609,654

(22) PCT Filed: May 7, 2020
(86) PCT No.: PCT/US20/31927

§ 371 Date: Nov. 8, 2021

Related U.S. Application Data
(60) Provisional application No. 62/846,348, filed on May 10, 2019.

(51) Int. Cl. A61K 31/542; A61K 31/4375; A61K 31/4985; A61K 31/5383; A61K 31/55

(52) U.S. Cl. CPC A61K 31/542; A61K 31/4375; A61K 31/55; A61K 31/5383; A61K 31/4985

(57) ABSTRACT

Described herein are compounds that are useful in treating an alphavirus infection. In some embodiments, the alphavirus is the Chikungunya virus.

### CROSS-REFERENCE TO RELATED APPLICATIONS

This application claims the benefit of U.S. Provisional Application Ser. No. 62/846,348 filed May 10, 2019, which is hereby incorporated by reference in its entirety.

### STATEMENT AS TO FEDERALLY SPONSORED RESEARCH

This invention was made with government support under Contract number R43AI118115 and Contract number R44AI122421, awarded by the National Institutes of Health. The government has certain rights in the invention.

### BACKGROUND OF THE INVENTION

Chikungunya virus (CHIKV) is a mosquito-borne arbovirus that causes self-limiting febrile illness in humans. A significant fraction of patients, however, experience long term sequelae including intense arthralgia and musculoskeletal pain that can persist for several years. Currently, there are no antiviral agents or vaccines approved for the treatment or prevention of CHIKV-related disease.

### BRIEF SUMMARY OF THE INVENTION

Disclosed herein are methods for treating an alphavirus infection comprising the step of administering to a subject in need thereof a compound of Formula (I), or a pharmaceutically acceptable salt, solvate, or stereoisomer thereof:

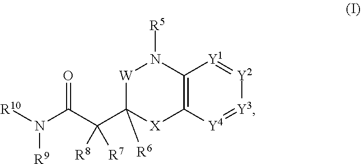

wherein:
X is —S—, —O—, —NR^{X}—, or —CR^{X1}R^{X2}—;
R^{X} is hydrogen, —S(═O)R^{a}, —S(═O)₂R^{a}, —S(═O)₂NR^{b}W, -C(═O)R^{a}, —C(═O)OR^{b}, —C(═O)NR^{b}R^{c}, C₁-C₆alkyl, C₁-C₆deuteroalkyl, or C₁-C₆haloalkyl;
R^{X1} and R^{X2} are independently hydrogen, deuterium, halogen, —CN, —OH, —OR^{a}, —NR^{b}R^{c}, —C(═O)R^{a}, C₁-C₆alkyl, C₁-C₆deuteroalkyl, or C₁-C₆haloalkyl;
W is >C═O, >C═S, or —CR^{W1}R^{W2}—;
R^{W1} and R^{W2} are independently hydrogen, deuterium, halogen, —CN, —OH, —OR^{a}, —NR^{b}R^{c}, —C(═O)R^{a}, C₁-C₆alkyl, C₁-C₆deuteroalkyl, or C₁-C₆haloalkyl;
Y¹ is N or CR¹;
Y² is N or CR²;
Y³ is N or CR³;
Y⁴ is N or CR⁴;
R¹, R², R³, and R⁴ are independently hydrogen, deuterium, halogen, —CN, —OH, —OR^{a}, —NO₂, —NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
R⁵ is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆deuteroalkyl, or C₁-C₆haloalkyl;
R⁶ is hydrogen, deuterium, halogen, —CN, —OH, —NR^{b}R^{c}, C₁-C₆alkyl, C₁-C₆deuteroalkyl, or C₁-C₆haloalkyl;
R⁷ and R⁸ are independently hydrogen, deuterium, halogen, —CN, —OH, —OR^{a}, —NR^{b}R^{c}, —C(═O)R^{a}, C₁-C₆alkyl, C₁-C₆deuteroalkyl, or C₁-C₆haloalkyl;
R⁹ is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆deuteroalkyl, or C₁-C₆haloalkyl;
R¹⁰ is C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl are optionally substituted with one, two, or three R^{10a};
or R⁹ and R¹⁰ are taken together to form a heterocycloalkyl ring optionally substituted with one, two, or three R^{10b};
each R^{10a} is independently deuterium, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, -C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; or two R¹⁰a on the same carbon are taken together to form an oxo;
each R^{10b} is independently deuterium, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{b}, —C(═O)OR^{b}, —OC(═O)OR^{b}, -C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; or two R¹⁰b on the same carbon are taken together to form an oxo;
each R^{a} is independently C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{b} and R^{c} are independently hydrogen, deuterium, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{b} and R^{c} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Aliphatic chain” refers to a linear chemical moiety that is composed of only carbons and hydrogens. In some embodiments, the aliphatic chain is saturated. In some embodiments, the aliphatic chain is unsaturated. In some embodiments, the unsaturated aliphatic chain contains one unsaturation. In some embodiments, the unsaturated aliphatic chain contains more than one unsaturation. In some embodiments, the unsaturated aliphatic chain contains two unsaturations. In some embodiments, the unsaturated aliphatic chain contains one double bond. In some embodiments, the unsaturated aliphatic chain contains two double bonds.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C_{I}-Cₛ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-Cio alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6-to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclop[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl comprises 1 or 2 heteroatoms selected from nitrogen and oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroi sobenzofuran-1-yl, 3-oxo-1,3-dihydroi sobenzofuran-1-yl, methyl-2-oxo-1,3-dioxo1-4-yl, and 2-oxo-1,3-dioxol-4-yl. The term heterocycloalkyl also includes all ring forms of the carbohydrates, including but not limited to, the monosaccharides, the disaccharides and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl. Unless stated otherwise specifically in the specification, a Heteroalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “oxo” means ═O.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound disclosed herein being administered which will relieve to some extent one or more of the symptoms of the disease or condition being treated. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

### Compounds

Described herein are compounds that are useful in treating an alphavirus infection such as a Chikungunya virus infection.

Disclosed herein is a compound of Formula (I), or a pharmaceutically acceptable salt, solvate, or stereoisomer thereof:

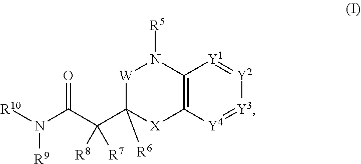

wherein:
X is —S—, —O—, —NR^{X}—, or —CR^{X1}R^{X2}—;
R^{X} is hydrogen, —S(═O)R^{a}, —S(═O)₂R^{a}, —S(═O)₂NR^{b}W, -C(═O)R^{a}, —C(═O)OR^{b}, —C(═O)NR^{b}R^{c}, C₁-C₆alkyl, C₁-C₆deuteroalkyl, or C₁-C₆haloalkyl;
R^{X1} and R^{X2} are independently hydrogen, deuterium, halogen, —CN, —OH, —OR^{a}, —NR^{b}R^{c}, —C(═O)R^{a}, C₁-C₆alkyl, C₁-C₆deuteroalkyl, or C₁-C₆haloalkyl;
W is >C═O, >C═S, or —CR^{W1}R^{W2}—;
R^{W1} and R^{W2} are independently hydrogen, deuterium, halogen, —CN, —OH, —OR^{a}, —NR^{b}R^{c}, —C(═O)R^{a}, C₁-C₆alkyl, C₁-C₆deuteroalkyl, or C₁-C₆haloalkyl;
Y¹ is N or CR¹;
Y² is N or CR²;
Y³ is N or CR³;
Y⁴ is N or CR⁴;
R¹, R², R³, and R⁴ are independently hydrogen, deuterium, halogen, —CN, —OH, —OR^{a}, —NO₂, —NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
R⁵ is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆deuteroalkyl, or C₁-C₆haloalkyl;
R⁶ is hydrogen, deuterium, halogen, —CN, —OH, —NR^{b}R^{c}, C₁-C₆alkyl, C₁-C₆deuteroalkyl, or C₁-C₆haloalkyl;
R⁷ and R⁸ are independently hydrogen, deuterium, halogen, —CN, —OH, —OR^{a}, —NR^{b}R^{c}, —C(═O)R^{a}, C₁-C₆alkyl, C₁-C₆deuteroalkyl, or C₁-C₆haloalkyl;
R⁹ is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl,
R¹⁰ is C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl are optionally substituted with one, two, or three R^{10a};
or R⁹ and R¹⁰ are taken together to form a heterocycloalkyl ring optionally substituted with one, two, or three R^{10b};
each R^{10a} is independently deuterium, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, -C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; or two R^{10a} on the same carbon are taken together to form an oxo;
each R^{10b} is independently deuterium, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{b}, —C(═O)OR^{b}, —OC(═O)OR^{b}, -C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; or two R¹⁰b on the same carbon are taken together to form an oxo;
each R^{a} is independently C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —(═O)Me, —C(═O)OH, —C(═O)OMe, C₁-C₆alkyl, or C₁-C₆haloalkyl; and
each R^{b} and R^{c} are independently hydrogen, deuterium, C₁-C₆alkyl, C₁-C₆deuteroalkyl, C₁-C₆haloalkyl,

C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{b} and R^{c} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

In some embodiments of a compound of Formula (I), X is —S—.

In some embodiments of a compound of Formula (I), X is —O—.

In some embodiments of a compound of Formula (I), X is —NR^{X}—.

In some embodiments of a compound of Formula (I), R^{X} is hydrogen or C₁-C₆alkyl. In some embodiments of a compound of Formula (I), R^{X} is hydrogen. In some embodiments of a compound of Formula (I), R^{X} is C₁-C₆alkyl.

In some embodiments of a compound of Formula (I), X is —CR^{X1}R^{X2}—.

In some embodiments of a compound of Formula (I), R^{X1} and R^{X2} are independently hydrogen, deuterium, halogen, C₁-C₆alkyl, C₁-C₆deuteroalkyl, or C₁-C₆haloalkyl. In some embodiments of a compound of Formula (I), R^{X1} and R^{X2} are independently hydrogen, deuterium, halogen, or C₁-C₆alkyl. In some embodiments of a compound of Formula (I), R^{X1} and R^{X2} are independently hydrogen or halogen. In some embodiments of a compound of Formula (I), R^{X1} and R^{X2} are hydrogen.

In some embodiments of a compound of Formula (I), Y¹ is N. In some embodiments of a compound of Formula (I), Y¹ is CR^{I}. In some embodiments of a compound of Formula (I), Y² is N. In some embodiments of a compound of Formula (I), Y² is CR². In some embodiments of a compound of Formula (I), Y³ is N. In some embodiments of a compound of Formula (I), Y³ is CR³. In some embodiments of a compound of Formula (I), Y⁴ is N. In some embodiments of a compound of Formula (I), Y⁴ is CR⁴.

In some embodiments of a compound of Formula (I), R¹, R², R³, and R⁴ are independently hydrogen, deuterium, halogen, —CN, —OH, —OR^{a}, —NR^{b}R^{c}, —C(═O)R^{a}, —C(═O)0R^{b}, —C(═O)NR^{b}Rc, C₁-C₆alkyl, C₁-C₆deuteroalkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I), R¹, R², R³, and R⁴ are independently hydrogen, deuterium, halogen, —CN, —OH, —OR^{a}, —NR^{b}R^{c}, C₁-C₆alkyl, C₁-C₆deuteroalkyl, or C₁-C₆haloalkyl. In some embodiments of a compound of Formula (I), R¹, R², R³, and R⁴ are independently hydrogen, deuterium, halogen, or C₁-C₆alkyl. In some embodiments of a compound of Formula (I), R¹, R², R³, and R⁴ are independently hydrogen or halogen. In some embodiments of a compound of Formula (I), R¹, R², R³, and R⁴ are hydrogen.

In some embodiments of a compound of Formula (I), W is >C═O.

In some embodiments of a compound of Formula (I), W is —CR^{W1}R^{W2}-.

In some embodiments of a compound of Formula (I), R^{W1} and R^{W2} are independently hydrogen, deuterium, halogen, C₁-C₆alkyl, C₁-C₆deuteroalkyl, or C₁-C₆haloalkyl. In some embodiments of a compound of Formula (I), R^{W1} and R^{W2} are independently hydrogen, deuterium, halogen, or C₁-C₆alkyl. In some embodiments of a compound of Formula (I), R^{W1} and R^{W2} are independently hydrogen, halogen, or C₁-C₆alkyl. In some embodiments of a compound of Formula (I), R^{W1} and R^{W2} are independently hydrogen or halogen. In some embodiments of a compound of Formula (I), R^{W1} and R^{W2} are hydrogen. In some embodiments of a compound of Formula (I), R⁵ is hydrogen, C₁-C₆alkyl, or C₁-C₆haloalkyl. In some embodiments of a compound of Formula (I), R⁵ is hydrogen or C₁-C₆alkyl. In some embodiments of a compound of Formula (I), R⁵ is hydrogen or C₁-C₆alkyl.

In some embodiments of a compound of Formula (I), R⁶ is hydrogen, deuterium, or halogen. In some embodiments of a compound of Formula (I), R⁶ is hydrogen.

In some embodiments of a compound of Formula (I), R⁷ and R⁸ are independently hydrogen, deuterium, halogen, C₁-C₆alkyl, or C₁-C₆haloalkyl. In some embodiments of a compound of Formula (I), R⁷ and R⁸ are independently hydrogen, halogen, or C₁-C₆alkyl. In some embodiments of a compound of Formula (I), R⁷ and R⁸ are hydrogen or C₁-C₆alkyl. In some embodiments of a compound of Formula (I), R⁷ and R⁸ are hydrogen.

In some embodiments of a compound of Formula (I), R⁹ is hydrogen, C₁-C₆alkyl, or C₁-C₆haloalkyl. In some embodiments of a compound of Formula (I), R⁹ is hydrogen or C₁-C₆alkyl. In some embodiments of a compound of Formula (I), R⁹ is hydrogen.

In some embodiments of a compound of Formula (I), R¹⁰ is C₁-C₆alkyl, C₁-C₆haloalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl are optionally substituted with one, two, or three R^{10a}. In some embodiments of a compound of Formula (I), R¹⁰ is C₁-C₆alkyl, C₁-C₆haloalkyl, cycloalkyl, C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl are optionally substituted with one, two, or three lea. In some embodiments of a compound of Formula (I), R¹⁰ is C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl are optionally substituted with one, two, or three R^{10a}.

In some embodiments of a compound of Formula (I), each R^{10a} is independently deuterium, halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl; or two R^{10a} on the same carbon are taken together to form an oxo. In some embodiments of a compound of Formula (I), each R^{10b} is independently deuterium, halogen, —OH, —OR^{a}, —NR^{b}R^{c}, C₁-C₆alkyl, or C₁-C₆haloalkyl; or two R^{10a} on the same carbon are taken together to form an oxo. In some embodiments of a compound of Formula (I), each R^{10a} is independently deuterium, halogen, —OH, —OR', —NR^{b}R^{c}, C₁-C₆alkyl, or C₁-C₆haloalkyl.

In some embodiments of a compound of Formula (I), R^{b} and R^{c} are taken together to form a heterocycloalkyl ring optionally substituted with one, two, or three R^{10b}.

In some embodiments of a compound of Formula (I), each R^{10b} is independently deuterium, halogen, —CN, —OH, —OR', —NR^{b}R^{c}, —C(═O)R^{a}, —C(═O)OR^{b}, —C(═O)NR^{b}R^{c}, C₁-C₆alkyl, C₁-C₆haloalkyl; or two R^{10b} on the same carbon are taken together to form an oxo. In some embodiments of a compound of Formula (I), each R^{10b} is independently halogen or C₁-C₆alkyl.

In some embodiments of a compound of Formula (I), each W is independently C₁-C₆alkyl, C₁-C₆haloalkyl, C₁-C₆deuteroalkyl, or cycloalkyl; wherein each alkyl and cycloalkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), each R^{10a} is independently C₁-C₆alkyl, C₁-C₆haloalkyl, or C₁-C₆deuteroalkyl. In some embodiments of a compound of Formula (I), each W is independently C₁-C₆alkyl.

In some embodiments of a compound of Formula (I), each R^{b} and R^{c} is independently hydrogen, C₁-C₆alkyl, C₁-C₆haloalkyl, C₁-C₆deuteroalkyl, or cycloalkyl; wherein each alkyl and cycloalkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), each R^{b} and R^{c} is independently hydrogen, C₁-C₆alkyl, C₁-C₆haloalkyl, or C₁-C₆deuteroalkyl. In some embodiments of a compound of Formula (I), each R^{b} and R^{c} is independently hydrogen or C₁-C₆alkyl. In some embodiments of a compound of Formula (I), each R^{b} and R^{c} is hydrogen. In some embodiments of a compound of Formula (I), each R^{b} and R^{c} is independently C₁-C₆alkyl.

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent. Labeled compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹ P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴ C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo42.2.21oct-2-ene-1-carboxylic acid, glucoheptonic acid, 4,4′-methylenebis-(3-hydroxy-2-ene-l-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involve the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

### Methods of Treatment

The compounds disclosed herein, or pharmaceutically acceptable salts, solvates, or stereoisomers thereof, are useful for the inhibition of alphaviruses.

Viruses of the genus Alphaviridae belong to the group IV Togaviridae family of viruses based on the well-known Baltimore taxonomic classification of viruses. Of the roughly 30 known alphaviruses, at least one third cause significant diseases in humans and animals worldwide, which manifest with such debilitating symptoms as encephalitis, arthritis, rashes, fevers, headache, nausea, myalgia, arthralgia (joint pain), arthropathy (diseases of the joint), chills, diarrhea, vomiting, lymphadenitis, malaise, and muscle soreness. In particular, Sindbis, Semliki Forest, O'nyong'nyong, Chikungunya, Mayaro, Ross River, Barmah Forest, Eastern Equine Encephalitis, Western Equine Encephalitis, and Venezuelan Equine Encephalitis viruses are medically relevant alphaviruses that generally infect human populations via insect vectors (e.g., mosquitoes) and can cause fatal encephalitis if alphaviral infection reaches the central nervous system (CNS). Neurotropic alphaviruses are also important members of the growing list of emerging or resurging public health threats (see, e.g., Gubler (2002) Arch. Med. Res. 33:330- 42) and are listed as CDC and NIAID category B bioterrorism agents due in part to numerous characteristics that make them potential biological weapons: (i) high clinical morbidity and mortality; (ii) potential for aerosol transmission; (iii) lack of effective countermeasures for disease prevention or control; (iv) public anxiety elicited by CNS infections; (v) ease with which large volumes of infectious materials can be produced; and (vi) potential for malicious introduction of foreign genes designed to increase alphavirus virulence (see, e.g., Sidwell et al. (2003) Antiviral Res. 57: 101-11).

Chikungunya virus (CHIKV) is a mosquito-borne Alphavirus endemic to Africa and Asia, which causes sudden onset of fever, rash, and debilitating poly-arthralgia in peripheral joints that can persist for years, particularly in older individuals. In 2013, CHIKV spread to the western hemisphere resulting in more than one million cases. Autochthonous transmission in the United States was confirmed in 2014. In response to the increased geographic distribution of CHIKV and the likelihood that elderly immune-naive populations may experience severe and life-threatening disease, a mouse model of age-related vulnerability to CHIKV infection was developed.

In certain embodiments, the present invention provides methods for treating an alphavirus infection in a subject, comprising administering to the subject an effective amount of a compound described herein. In some embodiments, the alphavirus is selected from the group consisting of Barmah Forest virus, Eastern equine encephalitis virus, Middelburg virus, Ndumu virus, Bebaru virus, Chikungunya virus, Getah virus, Mayaro virus, O'nyong'nyong virus, Ross River virus, Semliki Forest virus, Cabassou virus, Everglades virus, Mosso das Pedras virus, Mucambo virus, Paramana virus, Pixuna virus, Rio Negro virus, Trocara virus, Venezuelan equine encephalitis virus, Aura virus, Babanki virus, Kyzylagach virus, Sindbis virus, Ockelbo virus, Whataroa virus, Buggy Creek virus, Fort Morgan virus, Highlands J virus, Western equine encephalitis virus, Eilat virus, Mwinilunga alphavirus, Salmon pancreatic disease virus, Rainbow trout sleeping disease virus, Southern elephant seal virus, and Tonate virus. In some embodiments, the alphavirus is selected from the group consisting of Chikungunya virus, Mayaro virus, O'nyong'nyong virus, Venezuelan equine encephalitis virus, and Sindbis virus. In some embodiments, the alphavirus is the Chikungunya virus.

### Combination Therapy

In certain instances, the compound described herein, or a pharmaceutically acceptable salt, solvate, or stereoisomer thereof, is administered in combination with a second therapeutic agent.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, or stereoisomer thereof, is co-administered with a second therapeutic agent, wherein the compound described herein, or a pharmaceutically acceptable salt, solvate, or stereoisomer thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of the compounds disclosed herein will be utilized in formulating a pharmaceutical composition and/or in treatment regimens when the compounds disclosed herein are administered in combination with a second therapeutic agent. Therapeutically-effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, or stereoisomer thereof, as well as combination therapies, are administered before, during, or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, the compound of described herein, or a pharmaceutically acceptable salt, solvate, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

### EXAMPLES

### Example A: Synthesis of N-(cyclohexylmethyl)-2-(2-oxo-2,3-dihydro-1H-pyrido[2,3-b][1,4]thiazin-3-yl)acetamide

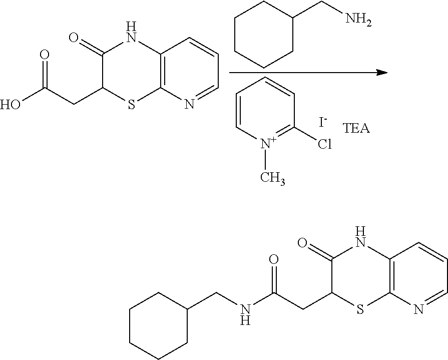

To the acid, 2-(2-oxo-2,3-dihydro-1H-pyrido[2,3-b][1,4]thiazin-3-ypacetic acid (available from ChemDiv, Inc., reference: Japan Patent JP63008387 A, Jan 14, 1988) (180 mg, 0.8 mmol) in DCM (16 mL) was added cyclohexanemethylamine (83 mg, 0.73 mmol) followed by triethylamine (0.37 mL, 2.65 mmol) and 2-chloro-1-methylpyridinium iodide (225 mg, 0.88 mmol). The reaction mixture was stirred at RT overnight, filtered. The solid was collected, washed with DCM and dried in vacuo to yield the title compound, 160 mg. ESI-MS m/z 320 (MH)⁺. ¹H NMR (300 MHz, DMSO-d6)δ 10.7 (s, 1H), 8.1 (m, 1H), 7.93 (m, 1H), 7.22 (m, 2H), 4.0 (m, 1H), 2.86 (m, 2H), 2.72 (m, 1H), 2.38 (m, 1H), 1.6 (m, 5H), 0.8-1.3 (m, 6H).

The following compounds in Table 1 were made according to the synthetic procedure described above using the appropriate amines as the starting materials.

TABLE 1

| | | | ESI-MS |
|---|---|---|---|
| Ex. | Structure | Chemical Name | (M + H)⁺ (m/z) |
| 1 | 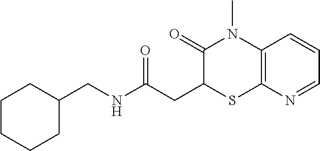 [SMILES: CN1C(=O)C(CC(=O)NCC2CCCCC2)Sc2ncccc21] | N-(cyclohexylmethyl)-2-(1- methyl-2-oxo-2,3-dihydro-1H- pyrido[2,3-b][1,4]thiazin-3- yl)acetamide | 334 |
| 2 | 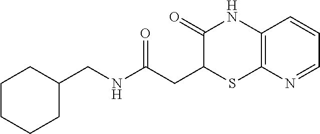 [SMILES: O=C(CC1Sc2ncccc2NC1=O)NCC1CCCCC1] | N-(cyclohexylmethyl)-2-(2- oxo-2,3-dihydro-1H- pyrido[2,3-b][1,4]thiazin-3- yl)acetamide | 320 |
| 3 | 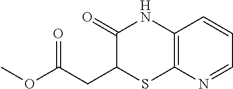 [SMILES: COC(=O)CC1Sc2ncccc2NC1=O] | methyl 2-(2-oxo-2,3-dihydro- 1H-pyrido[2,3-b][1,4]thiazin- 3-yl)acetate | 239 |
| 4 | 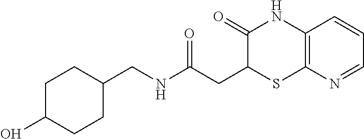 [SMILES: O=C(CC1Sc2ncccc2NC1=O)NCC1CCC(O)CC1] | N-((4- hydroxycyclohexyl)methyl)-2- (2-oxo-2,3-dihydro-1H- pyrido[2,3-b][1,4]thiazin-3- yl)acetamide | 336 |
| 5 | 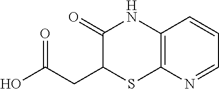 [SMILES: O=C(O)CC1Sc2ncccc2NC1=O] | 2-(2-oxo-2,3-dihydro-1H- pyrido[2,3-b][1,4]thiazin-3- yl)acetic acid | 225 |
| 6 | 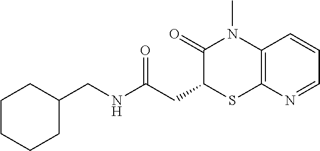 [SMILES: CN1C(=O)[C@@H](CC(=O)NCC2CCCCC2)Sc2ncccc21] | (R)-N-(cyclohexylmethyl)-2- (1-methyl-2-oxo-2,3-dihydro- 1H-pyrido[2,3-b][1,4]thiazin- 3-yl)acetamide | 334 |
| 7 | 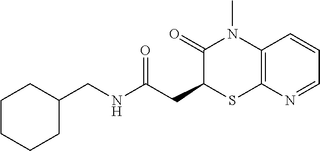 [SMILES: CN1C(=O)[C@H](CC(=O)NCC2CCCCC2)Sc2ncccc21] | (S)-N-(cyclohexylmethyl)-2- (1-methyl-2-oxo-2,3-dihydro- 1H-pyrido[2,3-b][1,4]thiazin- 3-yl)acetamide | 334 |
| 8 | 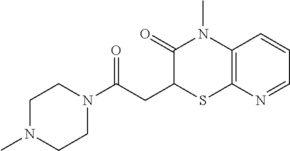 [SMILES: CN1CCN(C(=O)CC2Sc3ncccc3N(C)C2=O)CC1] | 1-methyl-3-(2-(4- methylpiperazin-1-yl)-2- oxoethyl)-1H-pyrido[2,3- b][1,4]thiazin-2(3H)-one | 321 |
| 9 | 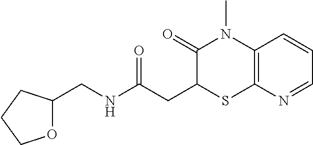 [SMILES: CN1C(=O)C(CC(=O)NCC2CCCO2)Sc2ncccc21] | 2-(1-methyl-2-oxo-2,3- dihydro-1H-pyrido[2,3- b][1,4]thiazin-3-yl)-N- ((tetrahydrofuran-2- yl)methyl)acetamide | 322 |
| 10 | 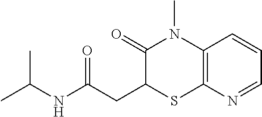 [SMILES: CC(C)NC(=O)CC1Sc2ncccc2N(C)C1=O] | N-isopropyl-2-(1-methyl-2- oxo-2,3-dihydro-1H- pyrido[2,3-b][1,4]thiazin-3- yl)acetamide | 280 |
| 11 | 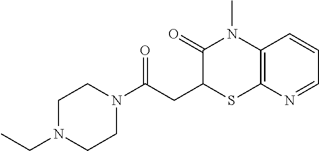 [SMILES: CCN1CCN(C(=O)CC2Sc3ncccc3N(C)C2=O)CC1] | 3-(2-(4-ethylpiperazin-1-yl)-2- oxoethyl)-1-methyl-1H- pyrido[2,3-b][1,4]thiazin- 2(3H)-one | 335 |
| 12 | 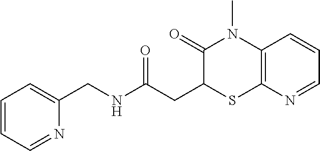 [SMILES: CN1C(=O)C(CC(=O)NCc2ccccn2)Sc2ncccc21] | 2-(1-methyl-2-oxo-2,3- dihydro-1H-pyrido[2,3- b][1,4]thiazin-3-yl)-N- (pyridin-2-ylmethyl)acetamide | 329 |
| 13 | 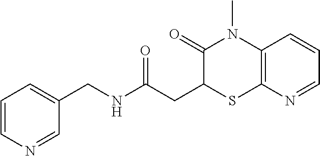 [SMILES: CN1C(=O)C(CC(=O)NCc2cccnc2)Sc2ncccc21] | 2-(1-methyl-2-oxo-2,3- dihydro-1H-pyrido[2,3- b][1,4]thiazin-3-yl)-N- (pyridin-3-ylmethyl)acetamide | 329 |
| 14 | 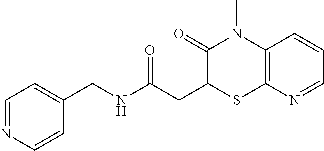 [SMILES: CN1C(=O)C(CC(=O)NCc2ccncc2)Sc2ncccc21] | 2-(1-methyl-2-oxo-2,3- dihydro-1H-pyrido[2,3- b][1,4]thiazin-3-yl)-N- (pyridin-4-ylmethyl)acetamide | 329 |
| 15 | 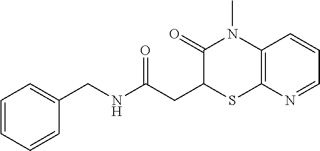 [SMILES: CN1C(=O)C(CC(=O)NCc2ccccc2)Sc2ncccc21] | N-benzyl-2-(1-methyl-2-oxo- 2,3-dihydro-1H-pyrido[2,3- b][1,4]thiazin-3-yl)acetamide | 328 |
| 16 | 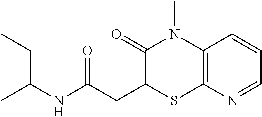 [SMILES: CCC(C)NC(=O)CC1Sc2ncccc2N(C)C1=O] | N-(sec-butyl)-2-(1-methyl-2- oxo-2,3-dihydro-1H- pyrido[2,3-b][1,4]thiazin-3- y)acetamide | 294 |
| 17 | 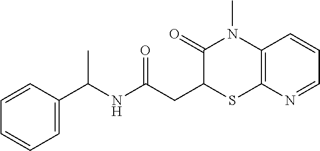 [SMILES: CC(NC(=O)CC1Sc2ncccc2N(C)C1=O)c1ccccc1] | 2-(1-methyl-2-oxo-2,3- dihydro-1H-pyrido[2,3- b][1,4]thiazin-3-yl)-N-(1- phenylethyl)acetamide | 342 |
| 18 | 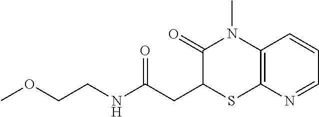 [SMILES: COCCNC(=O)CC1Sc2ncccc2N(C)C1=O] | N-(2-methoxyethyl)-2-(1- methyl-2-oxo-2,3-dihydro-1H- pyrido[2,3-b][1,4]thiazin-3- yl)acetamide | 296 |
| 19 | 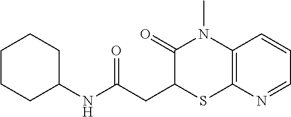 [SMILES: CN1C(=O)C(CC(=O)NC2CCCCC2)Sc2ncccc21] | N-cyclohexyl-2-(1-methyl-2- oxo-2,3-dihydro-1H- pyrido[2,3-b][1,4]thiazin-3- yl)acetamide | 320 |
| 20 | 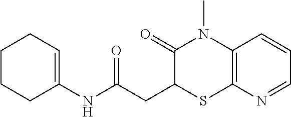 [SMILES: CN1C(=O)C(CC(=O)NC2=CCCCC2)Sc2ncccc21] | N-(cyclohex-1-en-1-yl)-2-(1- methyl-2-oxo-2,3-dihydro-1H- pyrido[2,3-b][1,4]thiazin-3- yl)acetamide | 318 |
| 21 | 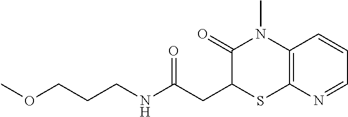 [SMILES: COCCCNC(=O)CC1Sc2ncccc2N(C)C1=O] | N-(3-methoxypropyl)-2-(1- methyl-2-oxo-2,3-dihydro-1H- pyrido[2,3-b][1,4]thiazin-3- yl)acetamide | 310 |
| 22 | 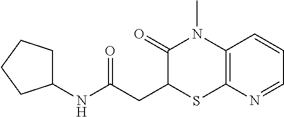 [SMILES: CN1C(=O)C(CC(=O)NC2CCCC2)Sc2ncccc21] | N-cyclopentyl-2-(1-methyl-2- oxo-2,3-dihydro-1H- pyrido[2,3-b][1,4]thiazin-3- yl)acetamide | 306 |
| 23 | 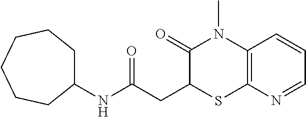 [SMILES: CN1C(=O)C(CC(=O)NC2CCCCCC2)Sc2ncccc21] | N-cycloheptyl-2-(1-methyl-2- oxo-2,3-dihydro-1H- pyrido[2,3-b][1,4]thiazin-3- yl)acetamide | 334 |
| 24 | 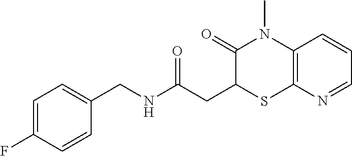 [SMILES: CN1C(=O)C(CC(=O)NCc2ccc(F)cc2)Sc2ncccc21] | N-(4-fluorobenzyl)-2-(1- methyl-2-oxo-2,3-dihydro-1H- pyrido[2,3-b][1,4]thiazin-3- yl)acetamide | 346 |
| 25 | 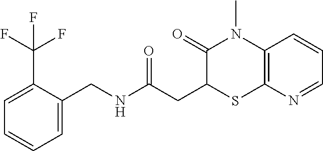 [SMILES: CN1C(=O)C(CC(=O)NCc2ccccc2C(F)(F)F)Sc2ncccc21] | 2-(1-methyl-2-oxo-2,3- dihydro-1H-pyrido[2,3- b][1,4]thiazin-3-yl)-N-(2- (trifluoromethyl)benzyl)aceta- mide | 396 |
| 26 | 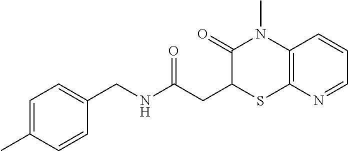 [SMILES: Cc1ccc(CNC(=O)CC2Sc3ncccc3N(C)C2=O)cc1] | 2-(1-methyl-2-oxo-2,3- dihydro-1H-pyrido[2,3- b][1,4]thiazin-3-yl)-N-(4- methylbenzyl)acetamide | 342 |
| 27 | 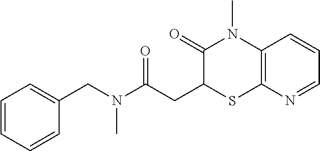 [SMILES: CN(Cc1ccccc1)C(=O)CC1Sc2ncccc2N(C)C1=O] | N-benzyl-N-methyl-2-(1- methyl-2-oxo-2,3-dihydro-1H- pyrido[2,3-b][1,4]thiazin-3- yl)acetamide | 342 |
| 28 | 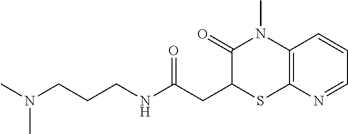 [SMILES: CN(C)CCCNC(=O)CC1Sc2ncccc2N(C)C1=O] | N-(3-(dimethylamino)propyl)- 2-(1-methyl-2-oxo-2,3- dihydro-1H-pyrido[2,3- b][1,4]thiazin-3-yl)acetamide | 323 |
| 29 | 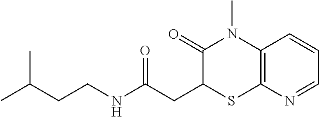 [SMILES: CC(C)CCNC(=O)CC1Sc2ncccc2N(C)C1=O] | N-isopentyl-2-(1-methyl-2- oxo-2,3-dihydro-1H- pyrido[2,3-b][1,4]thiazin-3- yl)acetamide | 308 |
| 30 | 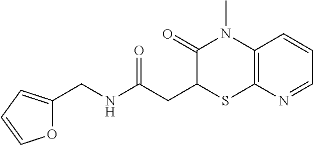 [SMILES: CN1C(=O)C(CC(=O)NCc2ccco2)Sc2ncccc21] | N-(furan-2-ylmethyl)-2-(1- methyl-2-oxo-2,3-dihydro-1H- pyrido[2,3-b][1,4]thiazin-3- yl)acetamide | 318 |
| 31 | 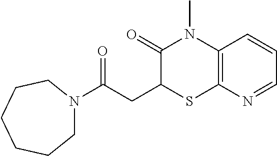 [SMILES: CN1C(=O)C(CC(=O)N2CCCCCC2)Sc2ncccc21] | 3-(2-(azepan-1-yl)-2- oxoethyl)-1-methyl-1H- pyrido[2,3-b][1,4]thiazin- 2(3H)-one | 320 |
| 32 | 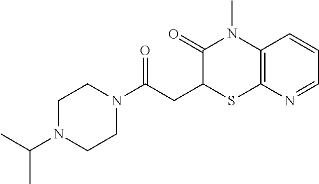 [SMILES: CC(C)N1CCN(C(=O)CC2Sc3ncccc3N(C)C2=O)CC1] | 3-(2-(4-isopropylpiperazin-1- yl)-2-oxoethyl)-1-methyl-1H- pyrido[2,3-b][1,4]thiazin- 2(3H)-one | 349 |
| 33 | 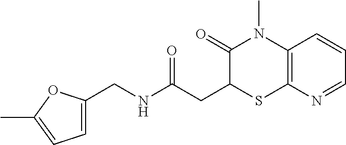 [SMILES: Cc1ccc(CNC(=O)CC2Sc3ncccc3N(C)C2=O)o1] | 2-(1-methyl-2-oxo-2,3- dihydro-1H-pyrido[2,3- b][1,4]thiazin-3-yl)-N-((5- methylfuran-2- yl)methyl)acetamide | 332 |
| 34 | 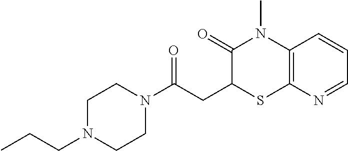 [SMILES: CCCN1CCN(C(=O)CC2Sc3ncccc3N(C)C2=O)CC1] | 1-methyl-3-(2-oxo-2-(4- propylpiperazin-1-yl)ethyl)- 1H-pyrido[2,3-b][1,4]thiazin- 2(3H)-one | 349 |
| 35 | 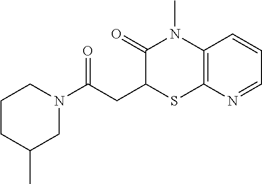 [SMILES: CC1CCCN(C(=O)CC2Sc3ncccc3N(C)C2=O)C1] | 1-methyl-3-(2-(3- methylpiperidin-1-yl)-2- oxoethyl)-1H-pyrido[2,3- b][1,4]thiazin-2(3H)-one | 320 |
| 36 | 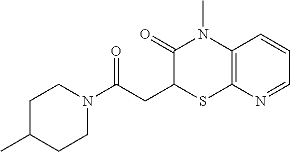 [SMILES: CC1CCN(C(=O)CC2Sc3ncccc3N(C)C2=O)CC1] | 1-methyl-3-(2-(4- methylpiperidin-1-yl)-2- oxoethyl)-1H-pyrido[2,3- b][1,4]thiazin-2(3H)-one | 320 |
| 37 | 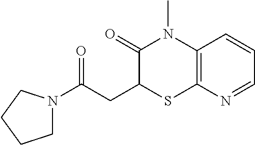 [SMILES: CN1C(=O)C(CC(=O)N2CCCC2)Sc2ncccc21] | 1-methyl-3-(2-oxo-2- (pyrrolidin-1-yl)ethyl)-1H- pyrido[2,3-b][1,4]thiazin- 2(3H)-one | 292 |
| 38 | 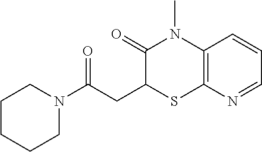 [SMILES: CN1C(=O)C(CC(=O)N2CCCCC2)Sc2ncccc21] | 1-methyl-3-(2-oxo-2- (piperidin-1-yl)ethyl)-1H- pyrido[2,3-b][1,4]thiazin- 2(3H)-one | 306 |
| 39 | 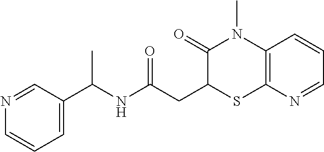 [SMILES: CC(NC(=O)CC1Sc2ncccc2N(C)C1=O)c1cccnc1] | 2-(1-methyl-2-oxo-2,3- dihydro-1H-pyrido[2,3- b][1,4]thiazin-3-yl)-N-(1- (pyridin-3-yl)ethyl)acetamide | 343 |
| 40 | 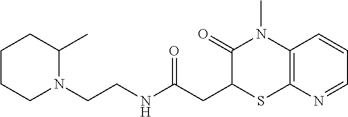 [SMILES: CC1CCCCN1CCNC(=O)CC1Sc2ncccc2N(C)C1=O] | 2-(1-methyl-2-oxo-2,3- dihydro-1H-pyrido[2,3- b][1,4]thiazin-3-yl)-N-(2-(2- methylpiperidin-1- yl)ethyl)acetamide | 363 |
| 41 | 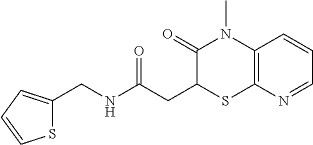 [SMILES: CN1C(=O)C(CC(=O)NCc2cccs2)Sc2ncccc21] | 2-(1-methyl-2-oxo-2,3- dihydro-1H-pyrido[2,3- b][1,4]thiazin-3-yl)-N- (thiophen-2- ylmethyl)acetamide | 334 |
| 42 | 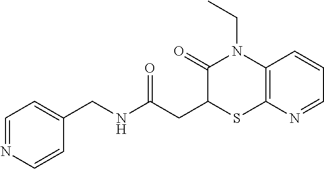 [SMILES: CCN1C(=O)C(CC(=O)NCc2ccncc2)Sc2ncccc21] | 2-(1-ethyl-2-oxo-2,3-dihydro- 1H-pyrido[2,3-b][1,4]thiazin- 3-yl)-N-(pyridin-4- ylmethyl)acetamide | 343 |
| 43 | 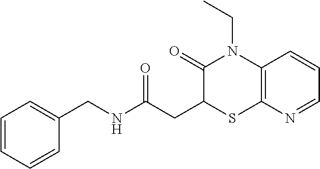 [SMILES: CCN1C(=O)C(CC(=O)NCc2ccccc2)Sc2ncccc21] | N-benzyl-2-(1-ethyl-2-oxo- 2,3-dihydro-1H-pyrido[2,3- b][1,4]thiazin-3-yl)acetamide | 342 |
| 44 | 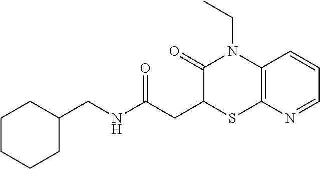 [SMILES: CCN1C(=O)C(CC(=O)NCC2CCCCC2)Sc2ncccc21] | N-(cyclohexylmethyl)-2-(1- ethyl-2-oxo-2,3-dihydro-1H- pyrido[2,3-b][1,4]thiazin-3- yl)acetamide | 348 |
| 45 | 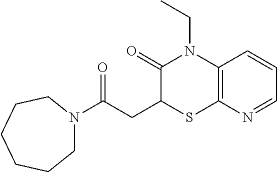 [SMILES: CCN1C(=O)C(CC(=O)N2CCCCCC2)Sc2ncccc21] | 3-(2-(azepan-1-yl)-2- oxoethyl)-1-ethyl-1H- pyrido[2,3-b][1,4]thiazin- 2(3H)-one | 334 |
| 46 | 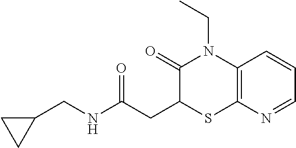 [SMILES: CCN1C(=O)C(CC(=O)NCC2CC2)Sc2ncccc21] | N-(cyclopropylmethyl)-2-(1- ethyl-2-oxo-2,3-dihydro-1H- pyrido[2,3-b][1,4]thiazin-3- yl)acetamide | 306 |
| 47 | 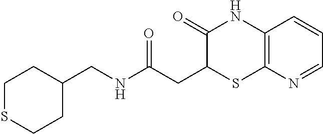 [SMILES: O=C(CC1Sc2ncccc2NC1=O)NCC1CCSCC1] | 2-(2-oxo-2,3-dihydro-1H- pyrido[2,3-b][1,4]thiazin-3- yl)-N-((tetrahydro-2H- thiopyran-4- yl)methyl)acetamide | 338 |
| 48 | 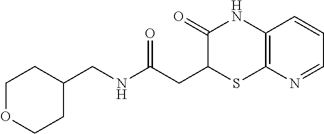 [SMILES: O=C(CC1Sc2ncccc2NC1=O)NCC1CCOCC1] | 2-(2-oxo-2,3-dihydro-1H- pyrido[2,3-b][1,4]thiazin-3- yl)-N-((tetrahydro-2H-pyran- 4-yl)methyl)acetamide | 322 |
| 49 | 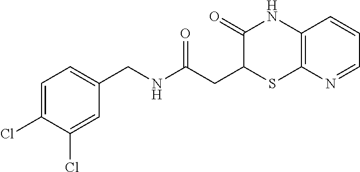 [SMILES: O=C(CC1Sc2ncccc2NC1=O)NCc1ccc(Cl)c(Cl)c1] | N-(3,4-dichlorobenzyl)-2-(2- oxo-2,3-dihydro-1H- pyrido[2,3-b][1,4]thiazin-3- yl)acetamide | 383 |
| 50 | 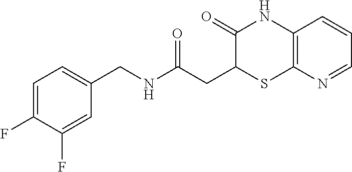 [SMILES: O=C(CC1Sc2ncccc2NC1=O)NCc1ccc(F)c(F)c1] | N-(3,4-difluorobenzyl)-2-(2- oxo-2,3-dihydro-1H- pyrido[2,3-b][1,4]thiazin-3- yl)acetamide | 350 |
| 51 | 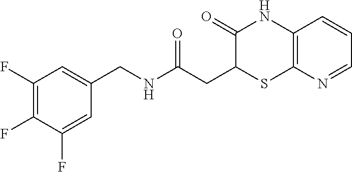 [SMILES: O=C(CC1Sc2ncccc2NC1=O)NCc1cc(F)c(F)c(F)c1] | 2-(2-oxo-2,3-dihydro-1H- pyrido[2,3-b][1,4]thiazin-3- yl)-N-(3,4,5- trifluorobenzyl)acetamide | 368 |
| 52 | 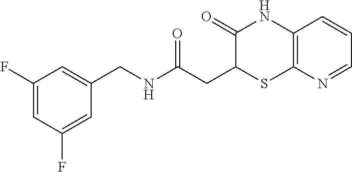 [SMILES: O=C(CC1Sc2ncccc2NC1=O)NCc1cc(F)cc(F)c1] | N-(3,5-difluorobenzyl)-2-(2- oxo-2,3-dihydro-1H- pyrido[2,3-b][1,4]thiazin-3- yl)acetamide | 350 |
| 53 | 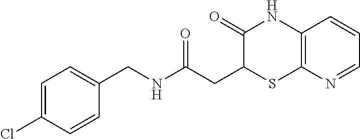 [SMILES: O=C(CC1Sc2ncccc2NC1=O)NCc1ccc(Cl)cc1] | N-(4-chlorobenzyl)-2-(2-oxo- 2,3-dihydro-1H-pyrido[2,3- b][1,4]thiazin-3-yl)acetamide | 349 |
| 54 | 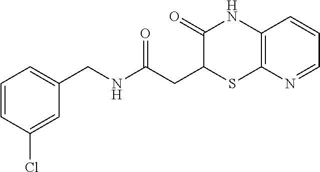 [SMILES: O=C(CC1Sc2ncccc2NC1=O)NCc1cccc(Cl)c1] | N-(3-chlorobenzyl)-2-(2-oxo- 2,3-dihydro-1H-pyrido[2,3- b][1,4]thiazin-3-yl)acetamide | 349 |
| 55 | 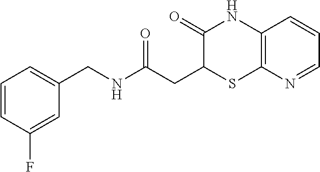 [SMILES: O=C(CC1Sc2ncccc2NC1=O)NCc1cccc(F)c1] | N-(3-fluorobenzyl)-2-(2-oxo- 2,3-dihydro-1H-pyrido[2,3- b][1,4]thiazin-3-yl)acetamide | 332 |
| 56 | 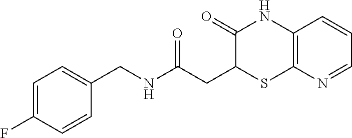 [SMILES: O=C(CC1Sc2ncccc2NC1=O)NCc1ccc(F)cc1] | N-(4-fluorobenzyl)-2-(2-oxo- 2,3-dihydro-1H-pyrido[2,3- b][1,4]thiazin-3-yl)acetamide | 332 |
| 57 | 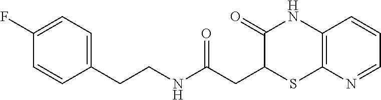 [SMILES: O=C(CC1Sc2ncccc2NC1=O)NCCc1ccc(F)cc1] | N-(4-fluorophenethyl)-2-(2- oxo-2,3-dihydro-1H- pyrido[2,3-b][1,4]thiazin-3- yl)acetamide | 346 |
| 58 | 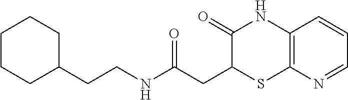 [SMILES: O=C(CC1Sc2ncccc2NC1=O)NCCC1CCCCC1] | N-(2-cyclohexylethyl)-2-(2- oxo-2,3-dihydro-1H- pyrido[2,3-b][1,4]thiazin-3- yl)acetamide | 334 |
| 59 | 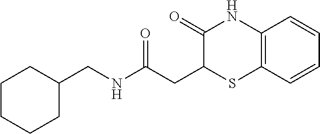 [SMILES: O=C(CC1Sc2ccccc2NC1=O)NCC1CCCCC1] | N-(cyclohexylmethyl)-2-(3- oxo-3,4-dihydro-2H- benzo[b][1,4]thiazin-2- yl)acetamide | 319 |
| 60 | 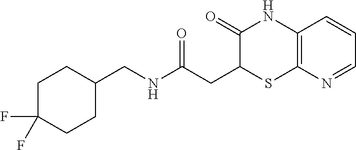 [SMILES: O=C(CC1Sc2ncccc2NC1=O)NCC1CCC(F)(F)CC1] | N-((4,4- difluorocyclohexyl)methyl)-2- (2-oxo-2,3-dihydro-1H- pyrido[2,3-b][1,4]thiazin-3- yl)acetamide | 356 |
| 61 | 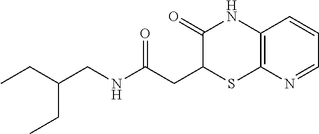 [SMILES: CCC(CC)CNC(=O)CC1Sc2ncccc2NC1=O] | N-(2-ethylbutyl)-2-(2-oxo-2,3- dihydro-1H-pyrido[2,3- b][1,4]thiazin-3-yl)acetamide | 308 |
| 62 | 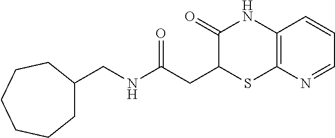 [SMILES: O=C(CC1Sc2ncccc2NC1=O)NCC1CCCCCC1] | N-(cycloheptylmethyl)-2-(2- oxo-2,3-dihydro-1H- pyrido[2,3-b][1,4]thiazin-3- yl)acetamide | 334 |
| 63 | 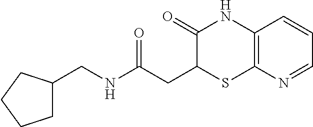 [SMILES: O=C(CC1Sc2ncccc2NC1=O)NCC1CCCC1] | N-(cyclopentylmethyl)-2-(2- oxo-2,3-dihydro-1H- pyrido[2,3-b][1,4]thiazin-3- yl)acetamide | 306 |
| 64 | 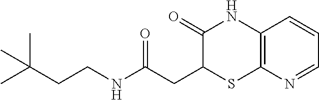 [SMILES: CC(C)(C)CCNC(=O)CC1Sc2ncccc2NC1=O] | N-(3,3-dimethylbutyl)-2-(2- oxo-2,3-dihydro-1H- pyrido[2,3-b][1,4]thiazin-3- yl)acetamide | 308 |
| 65 | 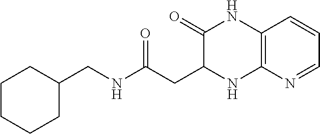 [SMILES: O=C(CC1Nc2ncccc2NC1=O)NCC1CCCCC1] | N-(cyclohexylmethyl)-2-(2- oxo-1,2,3,4- tetrahydropyrido[2,3- b]pyrazin-3-yl)acetamide | 303 |
| 66 | 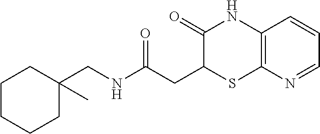 [SMILES: CC1(CNC(=O)CC2Sc3ncccc3NC2=O)CCCCC1] | N-((1- methylcyclohexyl)methyl)-2- (2-oxo-2,3-dihydro-1H- pyrido[2,3-b][1,4]thiazin-3- yl)acetamide | 334 |
| 67 | 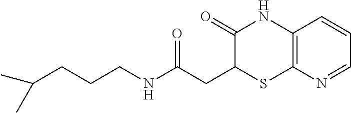 [SMILES: CC(C)CCCNC(=O)CC1Sc2ncccc2NC1=O] | N-(4-methylpentyl)-2-(2-oxo- 2,3-dihydro-1H-pyrido[2,3- b][1,4]thiazin-3-yl)acetamide | 308 |
| 68 | 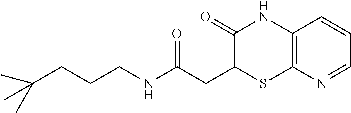 [SMILES: CC(C)(C)CCCNC(=O)CC1Sc2ncccc2NC1=O] | N-(4,4-dimethylpentyl)-2-(2- oxo-2,3-dihydro-1H- pyrido[2,3-b][1,4]thiazin-3- yl)acetamide | 322 |
| 69 | 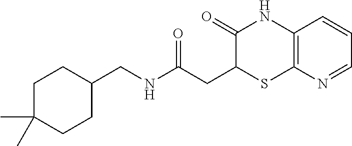 [SMILES: CC1(C)CCC(CNC(=O)CC2Sc3ncccc3NC2=O)CC1] | N-((4,4- dimethylcyclohexyl)methyl)- 2-(2-oxo-2,3-dihydro-1H- pyrido[2,3-b][1,4]thiazin-3- yl)acetamide | 348 |
| 70 | 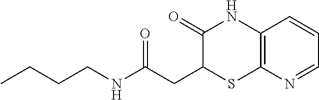 [SMILES: CCCCNC(=O)CC1Sc2ncccc2NC1=O] | N-butyl-2-(2-oxo-2,3-dihydro- 1H-pyrido[2,3-b][1,4]thiazin- 3-yl)acetamide | 280 |
| 71 | 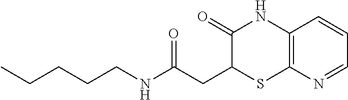 [SMILES: CCCCCNC(=O)CC1Sc2ncccc2NC1=O] | 2-(2-oxo-2,3-dihydro-1H- pyrido[2,3-b][1,4]thiazin-3- yl)-N-pentylacetamide | 294 |
| 72 | 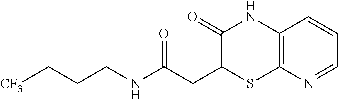 [SMILES: O=C(CC1Sc2ncccc2NC1=O)NCCCC(F)(F)F] | 2-(2-oxo-2,3-dihydro-1H- pyrido[2,3-b][1,4]thiazin-3- yl)-N-(4,4,4- trifluorobutyl)acetamide | 334 |
| 73 | 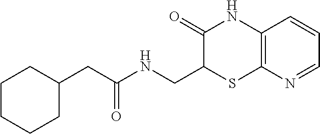 [SMILES: O=C(CC1CCCCC1)NCC1Sc2ncccc2NC1=O] | 2-cyclohexyl-N-((2-oxo-2,3- dihydro-1H-pyrido[2,3- b][1,4]thiazin-3- yl)methyl)acetamide | 320 |
| 74 | 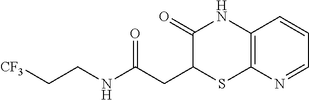 [SMILES: O=C(CC1Sc2ncccc2NC1=O)NCCC(F)(F)F] | 2-(2-oxo-2,3-dihydro-1H- pyrido[2,3-b][1,4]thiazin-3- yl)-N-(3,3,3- trifluoropropyl)acetamide | 320 |
| 75 | 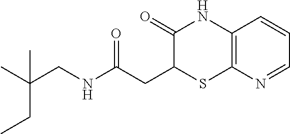 [SMILES: CCC(C)(C)CNC(=O)CC1Sc2ncccc2NC1=O] | N-(2,2-dimethylbutyl)-2-(2- oxo-2,3-dihydro-1H- pyrido[2,3-b][1,4]thiazin-3- yl)acetamide | 308 |
| 76 | 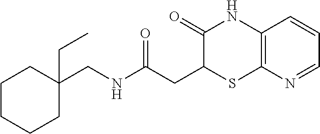 [SMILES: CCC1(CNC(=O)CC2Sc3ncccc3NC2=O)CCCCC1] | N-((1- ethylcyclohexyl)methyl)-2-(2- oxo-2,3-dihydro-1H- pyrido[2,3-b][1,4]thiazin-3- yl)acetamide | 348 |
| 77 | 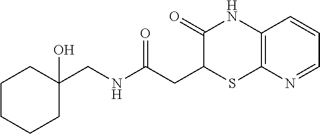 [SMILES: O=C(CC1Sc2ncccc2NC1=O)NCC1(O)CCCCC1] | N-((1- hydroxycyclohexyl)methyl)-2- (2-oxo-2,3-dihydro-1H- pyrido[2,3-b][1,4]thiazin-3- yl)acetamide | 336 |
| 78 | 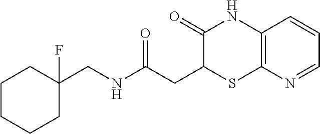 [SMILES: O=C(CC1Sc2ncccc2NC1=O)NCC1(F)CCCCC1] | N-((1- fluorocyclohexyl)methyl)-2- (2-oxo-2,3-dihydro-1H- pyrido[2,3-b][1,4]thiazin-3- yl)acetamide | 338 |
| 79 | 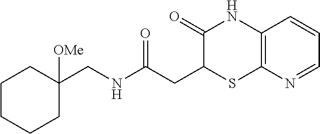 [SMILES: COC1(CNC(=O)CC2Sc3ncccc3NC2=O)CCCCC1] | N-((1- methoxycyclohexyl)methyl)- 2-(2-oxo-2,3-dihydro-1H- pyrido[2,3-b][1,4]thiazin-3- yl)acetamide | 350 |
| 80 | 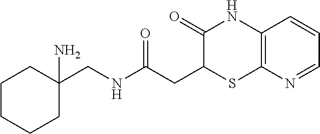 [SMILES: NC1(CNC(=O)CC2Sc3ncccc3NC2=O)CCCCC1] | N-((1- aminocyclohexyl)methyl)-2- (2-oxo-2,3-dihydro-1H- pyrido[2,3-b][1,4]thiazin-3- yl)acetamide | 335 |
| 81 | 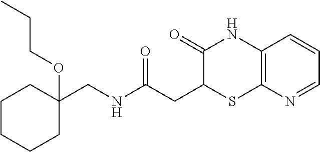 [SMILES: CCCOC1(CNC(=O)CC2Sc3ncccc3NC2=O)CCCCC1] | 2-(2-oxo-2,3-dihydro-1H- pyrido[2,3-b][1,4]thiazin-3- yl)-N-((1- propoxycyclohexyl)methyl)ace- tamide | 379 |
| 82 | 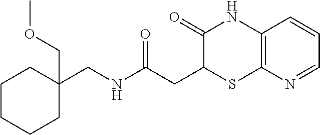 [SMILES: COCC1(CNC(=O)CC2Sc3ncccc3NC2=O)CCCCC1] | N-((1- (methoxymethyl)cyclohexyl)meth- yl)-2-(2-oxo-2,3-dihydro- 1H-pyrido[2,3-b][1,4]thiazin- 3-yl)acetamide | 364 |
| 83 | 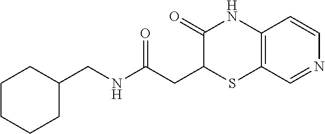 [SMILES: O=C(CC1Sc2cnccc2NC1=O)NCC1CCCCC1] | N-(cyclohexylmethyl)-2-(2- oxo-2,3-dihydro-1H- pyrido[3,4-b][1,4]thiazin-3- yl)acetamide | 320 |
| 84 | 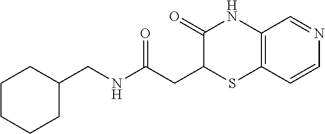 [SMILES: O=C(CC1Sc2ccncc2NC1=O)NCC1CCCCC1] | N-(cyclohexylmethyl)-2-(3- oxo-3,4-dihydro-2H- pyrido[4,3-b][1,4]thiazin-2- yl)acetamide | 320 |
| 85 | 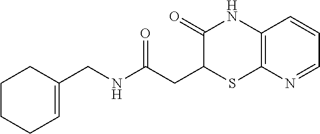 [SMILES: O=C(CC1Sc2ncccc2NC1=O)NCC1=CCCCC1] | N-(cyclohex-1-en-1- ylmethyl)-2-(2-oxo-2,3- dihydro-1H-pyrido[2,3- b][1,4]thiazin-3-yl)acetamide | 318 |
| 86 | 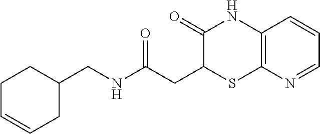 [SMILES: O=C(CC1Sc2ncccc2NC1=O)NCC1CC=CCC1] | N-(cyclohex-3-en-1- ylmethyl)-2-(2-oxo-2,3- dihydro-1H-pyrido[2,3- b][1,4]thiazin-3-yl)acetamide | 318 |
| 87 | 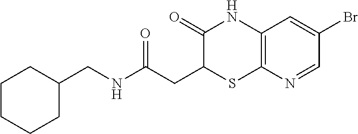 [SMILES: O=C(CC1Sc2ncc(Br)cc2NC1=O)NCC1CCCCC1] | 2-(7-bromo-2-oxo-2,3- dihydro-1H-pyrido[2,3- b][1,4]thiazin-3-yl)-N- (cyclohexylmethyl)acetamide | 399 |
| 88 | 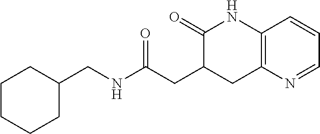 [SMILES: O=C(CC1Cc2ncccc2NC1=O)NCC1CCCCC1] | N-(cyclohexylmethyl)-2-(2- oxo-1,2,3,4-tetrahydro-1,5- naphthyridin-3-yl)acetamide | 302 |
| 89 | 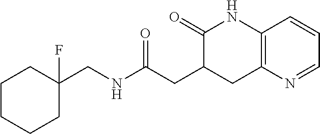 [SMILES: O=C(CC1Cc2ncccc2NC1=O)NCC1(F)CCCCC1] | N-((1- fluorocyclohexyl)methyl)-2- (2-oxo-1,2,3,4-tetrahydro-1,5- naphthyridin-3-yl)acetamide | 320 |
| 90 | 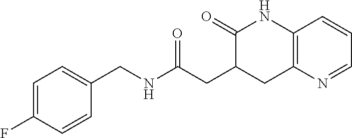 [SMILES: O=C(CC1Cc2ncccc2NC1=O)NCc1ccc(F)cc1] | N-(4-fluorobenzyl)-2-(2-oxo- 1,2,3,4-tetrahydro-1,5- naphthyridin-3-yl)acetamide | 314 |
| 91 | 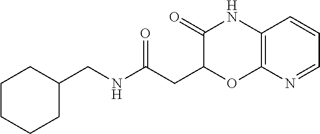 [SMILES: O=C(CC1Oc2ncccc2NC1=O)NCC1CCCCC1] | N-(cyclohexylmethyl)-2-(2- oxo-2,3-dihydro-1H- pyrido[2,3-b][1,4]oxazin-3- yl)acetamide | 304 |
| 92 | 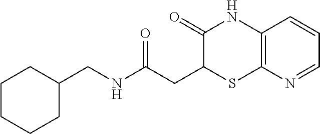 [SMILES: O=C(CC1Sc2ncccc2NC1=O)NCC1CCCCC1] | N-(cyclohexylmethyl)-2-(2,3- dihydro-1H-pyrido[2,3- b][1,4]thiazin-3-yl)acetamide | 306 |

### Example B: In vitro Assay

The compounds disclosed herein were tested against the vaccine strain CHIKV 181/clone25. CHIKV 181/clone25 was propagated in African green monkey kidney epithelial cells (Vero 76) obtained from ATCC [CRL-1587]. Vero cells were maintained in Eagle's minimal essential medium (EMEM) supplemented with 10% fetal bovine serum (FBS), 20 mM HEPES, pH 7.5 and 1× Penicillin/Streptomycin (Life Technologies) (complete medium). Chikungunya virus (CHIKV) strain 181/clone25 was generously provided by Dr. S. Weaver (University of Texas Medical Branch, Galveston, Tex.). Vero cells (2×10⁴ cells/well) were seeded into 96-well Cell Bind tissue culture plates (Corning) in 200 μL of complete medium. After 24 hours, tissue culture medium was removed and the monolayers were infected with CHIKV 181/clone25 at a multiplicity of infection (MOI)=0.01. Virus particles were diluted in EMEM supplemented with 2% FBS (assay medium) and allowed to adhere to Vero cells for 1 hr at 37° C. in a total volume of 30 μL. Following this period, 160 μL of assay medium was added to each well. For dose-response assays, various concentrations of inhibitors were subjected to ten 0.5Logio serial dilutions in DMSO. Compounds were then diluted stepwise 200-fold into assay medium to achieve the desired final concentration in 0.5% DMSO. After 48 hrs, live cells were fixed with 5% glutaraldehyde and stained with 0.5% crystal violet in 20% methanol. Cytopathic effect was quantified by evaluating absorbance at 595nm and protection from CPE was expressed as a percentage of the live cells in the non-infected control wells. The data was analyzed using the 4-parameter, non-linear regression model in GraphPad Prism. The EC₅₀ are shown in table 2. CHIKV EC₅₀: A>100 μM; B, 50-99.9 μM; C, 10-49.9 μM; D<10 μM.

TABLE 2

| Ex. | CHIKV EC₅₀ |
|---|---|
| 1 | D |
| 2 | D |
| 3 | A |
| 4 | A |
| 5 | A |
| 6 | D |
| 7 | C |
| 8 | A |
| 9 | A |
| 10 | A |
| 11 | A |
| 12 | A |
| 13 | A |
| 14 | A |
| 15 | C |
| 16 | A |
| 17 | A |
| 18 | A |
| 19 | A |
| 20 | C |
| 21 | A |
| 22 | B |
| 23 | C |
| 24 | D |
| 25 | A |
| 26 | C |
| 27 | A |
| 28 | A |
| 29 | D |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | C |
| 42 | A |
| 43 | A |
| 44 | C |
| 45 | A |
| 46 | A |
| 47 | C |
| 48 | A |
| 49 | A |
| 50 | C |
| 51 | B |
| 52 | A |
| 53 | C |
| 54 | A |
| 55 | A |
| 56 | D |
| 57 | C |
| 58 | C |
| 59 | A |
| 60 | C |
| 61 | C |
| 62 | C |
| 63 | D |
| 64 | A |
| 65 | A |
| 66 | D |
| 67 | C |
| 68 | C |
| 69 | C |
| 70 | B |
| 71 | C |
| 72 | A |
| 73 | C |
| 74 | A |
| 75 | D |
| 76 | D |
| 77 | D |
| 78 | D |
| 79 | D |
| 80 | A |
| 81 | C |
| 82 | B |
| 83 | A |
| 84 | A |
| 85 | C |
| 86 | D |
| 87 | C |
| 88 | C |
| 89 | C |
| 90 | C |
| 91 | C |
| 92 | C |

### Example C: Activity of Example 1 Against Other Viruses

Example 1 was tested against other alphaviruses and non-alphaviruses. The EC₅₀ are shown in tables 3 and 4. CHIKV EC₅₀: A >100 μM; B, 50-99.9 μM; C, 10-49.9 μM; D <10 μM.

TABLE 3

| Alphavirus | Complex | EC₅₀ (μM) |
|---|---|---|
| ONNV (SG-650) | SFV | D |
| MAYV | SFV | C |
| SINV (TR-339) | WEEV | A |
| VEEV (ZPC) | VEEV | D |

TABLE 4

| Virus | Family | EC₅₀ (μM) |
|---|---|---|
| Influenza | Orthomyxovirus | A |
| Dengue | Flavivirus | A |
| Vaccinia | Poxvirus | A |
| BK Polyomavirus | Polyomavirus | A |

The examples and embodiments described herein are for illustrative purposes only and in some embodiments, various modifications or changes are to be included within the purview of disclosure and scope of the appended claims.

What is claimed is:

1. A method for treating an alphavirus infection comprising the step of administering to a subject in need thereof a compound of Formula (I), or a pharmaceutically acceptable salt, solvate, or stereoisomer thereof: 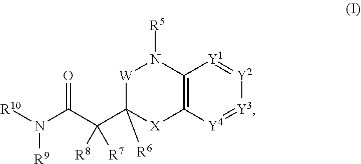
wherein:
X is —S—, —O—, —NR^{X}—, or —CR^{X1}R^{X2}—;
R^{X} is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆deuteroalkyl, or C₁-C₆haloalkyl;
R^{X1} and R^{X2} are independently hydrogen, deuterium, halogen, —CN, —OH, —OR^{a}, —NR^{b}R^{c}, —C(═O)R^{a}, C₁-C₆alkyl, C₁-C₆deuteroalkyl, or C₁-C₆haloalkyl;
W is >C═O, >C═S, or —CR^{W1}R^{W2}—;
R^{W1} and R^{W2} are independently hydrogen, deuterium, halogen, —CN, —OH, −OR^{a}, —NR^{b}R^{c}, —C(═O)R^{a}, C₁-C₆alkyl, C₁-C₆deuteroalkyl, or C₁-C₆haloalkyl;
Y¹ is N or CR¹;
Y² is N or CR²;
Y³ is N or CR³;
Y⁴ is N or CR⁴;
R¹, R², R³, and R⁴ are independently hydrogen, deuterium, halogen, —CN, —OH, —OR^{a}, —NO₂, —NR^{b}R^{c}, -C(═O)R^{a}, —OC(═O)R^{a}, —C(═O)OR^{b}, —OC(═O)OR^{b}, —C(═O)NR^{b}R^{c}, —OC(═O)NR^{b}R^{c}, -NR^{b}C(═O)NR^{b}R^{c}, —NR^{b}C(═O)R^{a}, —NR^{b}C(═O)0R^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
R⁵ is hydrogen, —S(═O)R^{a}, —S(═O)₂R^{a}, —S(═O)₂NR^{b}R^{c}, —C(═O)R^{a}, —C(═O)0R^{b}, —C(═O)NR^{b}R^{c}, C₁-C₆alkyl, C₁-C₆deuteroalkyl, or C₁-C₆haloalkyl;
R⁶ is hydrogen, deuterium, halogen, —CN, —OH, —OR^{a}, —NR^{b}R^{c}, C₁-C₆alkyl, C₁-C₆deuteroalkyl, or C₁-C₆haloalkyl;
R⁷ and R⁸ are independently hydrogen, deuterium, halogen, —CN, —OH, —OR^{a}, —NR^{b}R^{c}, —C(═O)1V, C₁-C₆alkyl, C₁-C₆deuteroalkyl, or C₁-C₆haloalkyl;
R⁹ is hydrogen, —S(═O)R^{a}, —S(═O)₂R^{a}, —S(═O)₂NR^{b}R^{c}, —C(═O)R^{a}, —C(═O)0R^{b}, —C(═O)NR^{b}R^{c}, C₁-C₆alkyl, C₁-C₆deuteroalkyl, or C₁-C₆haloalkyl;
R¹⁰ is C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl are optionally substituted with one, two, or three R^{10a};
or R⁹ and R¹⁰ are taken together to form a heterocycloalkyl ring optionally substituted with one, two, or three R^{10b};
each R^{10a} is independently deuterium, halogen, —CN, —OH, —SH, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, -C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; or two R^{10a} on the same carbon are taken together to form an oxo;
each R^{10b} is independently deuterium, halogen, —CN, —OH, —OR^{a}, —SH, —SW, —S(═O)R^{a}, —S(═O)₂R^{a}, —NO₂, —NR^{b}R^{c}, —NHS(═O)₂R^{a}, —S(═O)₂NR^{b}R^{c}, —C(═O)R¹, —OC(═O)R^{a}, —C(═O)OR^{b}, —OC(═O)OR^{b}, -C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; or two R^{10b} on the same carbon are taken together to form an oxo;
each R^{a} is independently C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{b} and R^{c} are independently hydrogen, deuterium, C₁-C₆alkyl, C₁-C₆deuteroalkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{b} and R^{c} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

2. The method of claim 1, wherein X is —S—.

3. The method of claim 1, wherein X is —O—.

4. The method of claim 1, wherein X is —NR^{X}—.

5. The method of claim 1 or 4, wherein R^{X} is hydrogen or C₁-C₆alkyl.

6. The method of claim 1, wherein X is —CR^{X1}R^{X2}—.

7. The method of claim 1 or 6, wherein R^{X1} and R^{X2} are hydrogen.

8. The method of any one of claims 1-7, wherein Y¹ is N.

9. The method of any one of claims 1-7, wherein Y¹ is CR¹.

10. The method of any one of claims 1-9, wherein Y² is N.

11. The method of any one of claims 1-9, wherein Y² is CR².

12. The method of any one of claims 1-11, wherein Y³ is N.

13. The method of any one of claims 1-11, wherein Y³ is CR³.

14. The method of any one of claims 1-13, wherein Y⁴ is N.

15. The method of any one of claims 1-13, wherein Y⁴ is CR⁴.

16. The method of any one of claim 1-7, 9, 11, 13, or 15, wherein R¹, R², R³, and R⁴ are independently hydrogen, deuterium, halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆deuteroalkyl, C₁-C₆haloalkyl, C₁-C₆hydroxyalkyl, C₁-C₆aminoalkyl, cycloalkyl, heterocycloalkyl, aryl, or heteroaryl.

17. The method of any one of claim 1-7, 9, 11, 13, 15, or 16, wherein R¹, R², R³, and R⁴ are independently hydrogen, deuterium, halogen, —CN, —OH, —OR^{a}, —NR^{b}R^{c}, C₁-C₆alkyl, C₁-C₆deuteroalkyl, or C₁-C₆haloalkyl.

18. The method of any one of claim 1-7, 9, 11, 13, or 15-17, wherein R¹, R², R³, and R⁴ are independently hydrogen, deuterium, halogen, or C₁-C₆alkyl.

19. The method of any one of claims 1-18, wherein W is >C═O.

20. The method of any one of claims 1-18, wherein W is —CR^{W1}R^{W2}—.

21. The method of any one of claim 1-18 or 20, wherein R^{W1} and R^{W2} are hydrogen.

22. The method of any one of claims 1-21, wherein R⁵ is hydrogen or C₁-C₆alkyl.

23. The method of any one of claims 1-22, wherein R⁶ is hydrogen.

24. The method of any one of claims 1-23, wherein R⁷ and R⁸ are hydrogen or C₁-C₆alkyl.

25. The method of any one of claims 1-24, wherein R⁷ and R⁸ are hydrogen.

26. The method of any one of claims 1-25, wherein R⁹ is hydrogen.

27. The method of any one of claims 1-26, wherein R¹⁰ is C₁-C₆alkyl, C₁-C₆haloalkyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl are optionally substituted with one, two, or three R^{10a}.

28. The method of any one of claims 1-27, wherein R¹⁰ is C₁-C₆alkyl, C₁-C₆haloalkyl, cycloalkyl, C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl are optionally substituted with one, two, or three R^{10a}.

29. The method of any one of claims 1-28, wherein R¹⁰ is C₁-C₆alkyl(cycloalkyl), C₁-C₆alkyl(heterocycloalkyl), C₁-C₆alkyl(aryl), or C₁-C₆alkyl(heteroaryl); wherein the alkyl, cycloalkyl, heterocycloalkyl, aryl, and heteroaryl are optionally substituted with one, two, or three R^{10a}.

30. The method of any one of claims 1-29, wherein each R^{10a} is independently deuterium, halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl; or two R^{10a} on the same carbon are taken together to form an oxo.

31. The method of any one of claims 1-29, wherein each R^{10a} is independently deuterium, halogen, —OH, —OR^{a}, —NR^{b}R^{c}, C₁-C₆alkyl, or C₁-C₆haloalkyl; or two R^{10a} on the same carbon are taken together to form an oxo.

32. The method of any one of claims 1-29, wherein each R^{10a} is independently deuterium, halogen, —OH, —OR^{a}, —NR^{b}R^{c}, C₁-C₆alkyl, or C₁-C₆haloalkyl.

33. The method of any one of claims 1-26, wherein R⁹ and R¹⁰ are taken together to form a heterocycloalkyl ring optionally substituted with one, two, or three R^{10b}.

34. The method of any one of claim 1-26 or 33, wherein each R^{10b} is independently deuterium, halogen, —CN, —OH, —OR^{a}, —NR^{b}R^{c}, —C(═O)R^{a}, —C(═O)OR^{b}, —C(═O)NR^{b}W, C₁-C₆alkyl, C₁-C₆haloalkyl; or two R^{um} on the same carbon are taken together to form an oxo.

35. The method of any one of claim 1-26 or 33 or 34, wherein each R^{10a} is independently halogen or C₁-C₆alkyl.

36. The method of claim 1, wherein the compound is selected from the group consisting of: 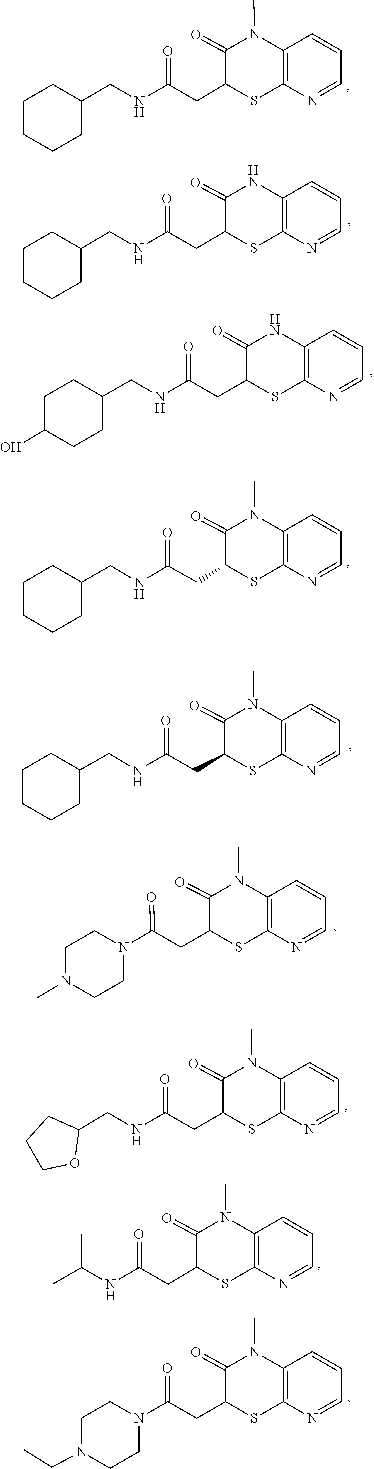 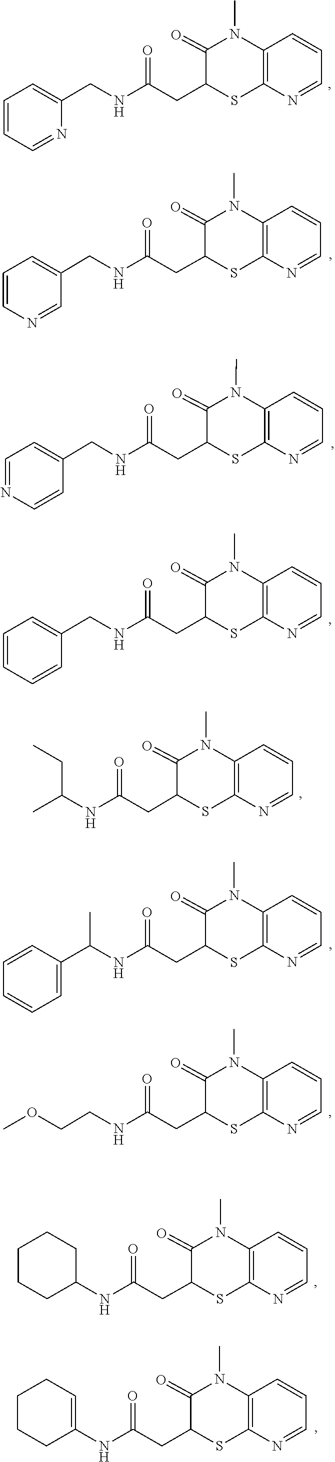 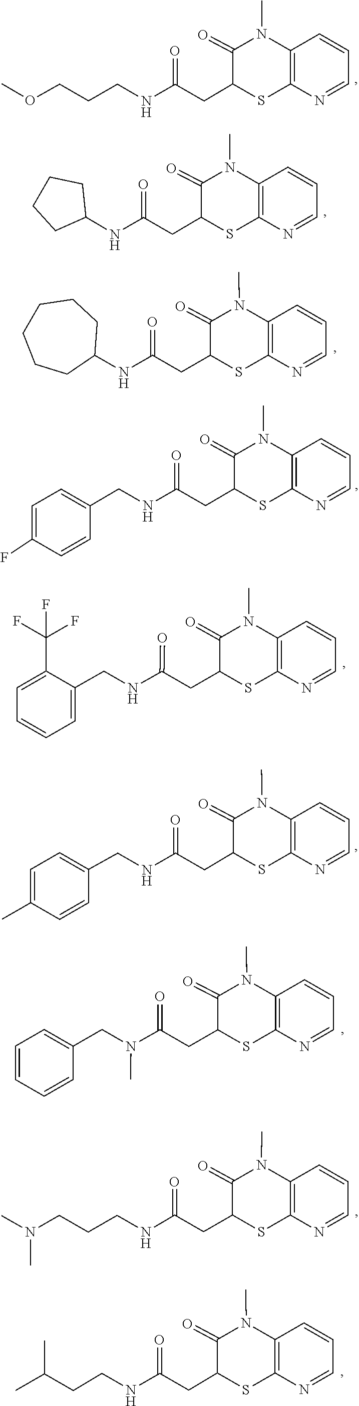 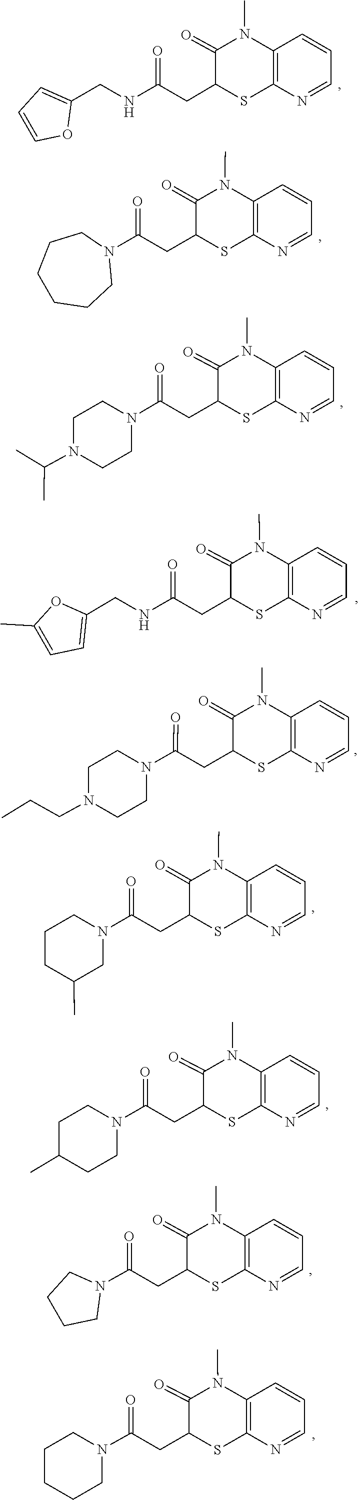 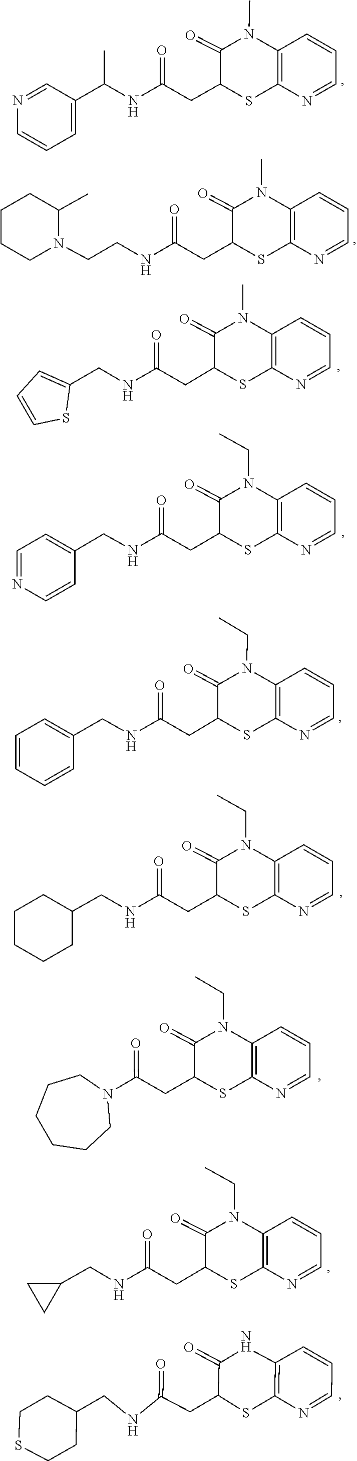 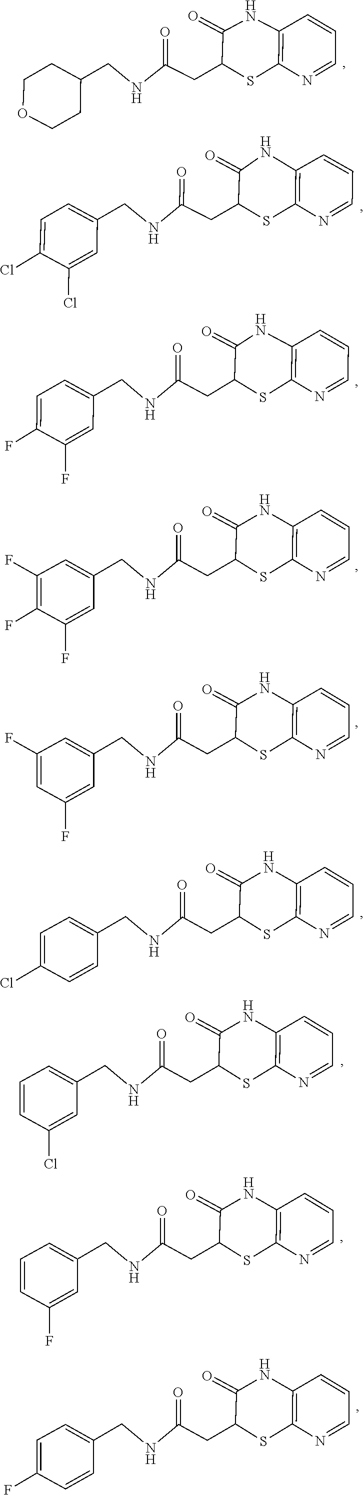 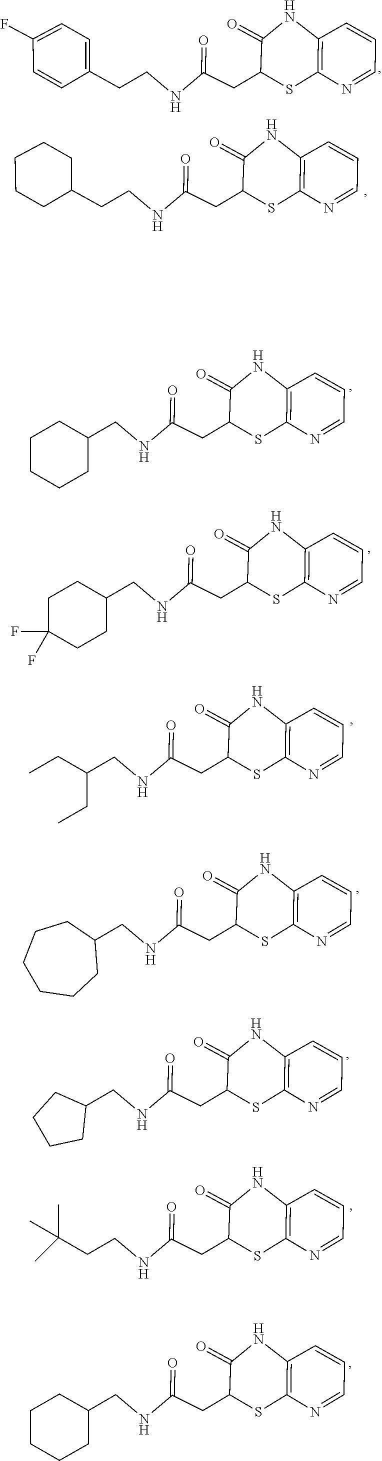 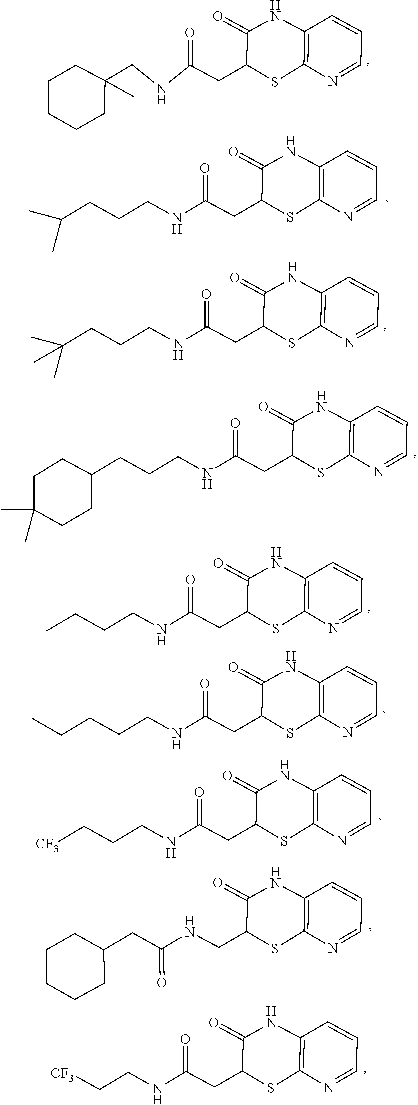 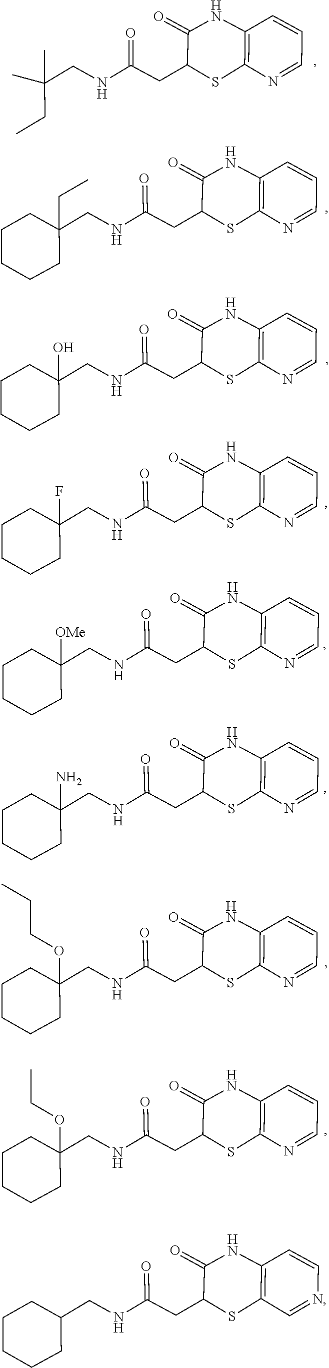 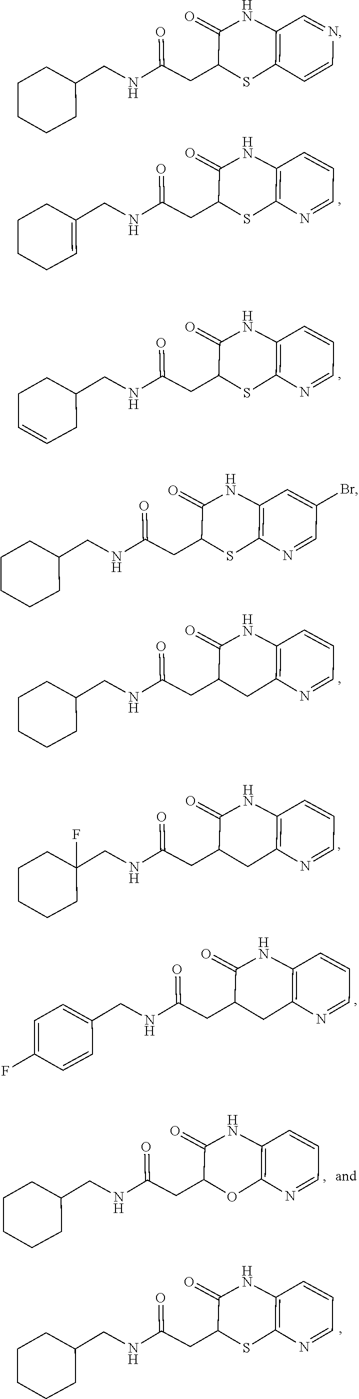
or
a pharmaceutically acceptable salt, solvate, or stereoisomer thereof.

37. The method of any one of claims 1-36, wherein the alphavirus is selected from the group consisting of Barmah Forest virus, Eastern equine encephalitis virus, Middelburg virus, Ndumu virus, Bebaru virus, Chikungunya virus, Getah virus, Mayaro virus, O'nyong'nyong virus, Ross River virus, Semliki Forest virus, Cabassou virus, Everglades virus, Mosso das Pedras virus, Mucambo virus, Paramana virus, Pixuna virus, Rio Negro virus, Trocara virus, Venezuelan equine encephalitis virus, Aura virus, Babanki virus, Kyzylagach virus, Sindbis virus, Ockelbo virus, Whataroa virus, Buggy Creek virus, Fort Morgan virus, Highlands J virus, Western equine encephalitis virus, Eilat virus, Mwinilunga alphavirus, Salmon pancreatic disease virus, Rainbow trout sleeping disease virus, Southern elephant seal virus, and Tonate virus.

38. The method of any one of claims 1-37, wherein the alphavirus is selected from the group consisting of Chikungunya virus, Mayaro virus, O'nyong'nyong virus, Venezuelan equine encephalitis virus, or Sindbis virus.

39. The method of any one of claims 1-38, wherein the alphavirus is the Chikungunya virus.
